(19) United States
(12) Patent Application Publication
(10) Pub. No.: US 2016/0287747 A1
(43) Pub. Date: Oct. 6, 2016

(54) SHAPED COLLAGEN TISSUE IMPLANT AND METHOD OF MANUFACTURING THEREOF

(71) Applicant: Mark Schallenberger, Bozeman, MT (US)

(72) Inventor: Mark Schallenberger, Bozeman, MT (US)

(21) Appl. No.: 15/087,553

(22) Filed: Mar. 31, 2016

Related U.S. Application Data
(60) Provisional application No. 62/140,761, filed on Mar. 31, 2015.

(51) Int. Cl. A61L 27/24

(52) U.S. Cl. CPC A61L 27/24; A61L 2430/00

(57) ABSTRACT

The invention relates to a shaped collagen tissue implant comprised of collagen molded in a particular shape and methods of manufacture thereof. The shaped tissue implants of the invention generate tissue structures that retain their size, shape, flexibility, and texture after implantation into a patient.

### CROSS REFERENCE TO RELATED APPLICATIONS

This application claims priority and the benefit under 35 U.S.C. §119(e) to U.S. Provisional Patent Application Ser. No. 62/140,761 filed Mar. 31, 2015, which is incorporated herein in its entirety by reference.

### FIELD OF THE INVENTION

The invention generally relates to shaped collagen implants of adjustable dimensions and methods for manufacturing the same.

### DESCRIPTION OF PRIOR ART

Collagen-based implants can be created by a variety of techniques. Commonly particulated collagen-based implants are used as fillers for a variety of surgical treatments. Collagen fillers are generally temporary in nature once implanted. In order to generate long-lasting shaped collagen-based implants, additional physical and/or chemical manipulations are performed. These manipulations can include chemical crosslinking, irradiation crosslinking, pressurization, and customized drying techniques. These manipulations, as described in the prior art, often retard the ability of the collagen implant to be integrated by a host following implantation. This lack of integration can adversely affect the useful lifetime of the implant. It is desirable for collagen-based tissue implants to be manufactured by simplified procedures and minimal chemical manipulations while retaining its shape, desired physical characteristics, and exhibits successful integration with host tissue on implantation.

U.S. Patent Publication No. 2014/0277577 to Garigapati (incorporated in its entirety by reference) discloses pepsinized collagen implants and methods of making the same. A preferred method of manufacturing a shaped collagen-based implant comprises the steps of digesting collagen with pepsin, solubilizing the pepsinized collagen in a polyol, drying the solubilized pepsinized collagen, and reacting the resultant collagen with a cross-linking agent.

U.S. Patent Publication No. 2012/0010728 to Sun et al. (incorporated in its entirety by reference) discloses methods for shaping tissue matrices without using chemical crosslinking agents. The overall method of preforming the tissue matrices into predefined shapes or configurations comprises the partially dehydrating a selected tissue matrix, applying mechanical forces to the tissue matrix, and exposing the tissue matrix to radiation.

U.S. Patent Publication No. 2014/0257516 to Mills et al. (incorporated in its entirety by reference) discloses a tissue matrix composition comprising shredded tissue combined with an aqueous carrier and dried in a predetermined shape. The method of making the matrix comprises the steps of defatting the tissue, lyophilizing the defatted tissue, shredding the lyophilized tissue, mixing the shredded tissue in an aqueous carrier to form a slurry, transferring the slurry to a mold, and drying the slurry.

The present invention discloses shaped collagen tissue implants and methods for manufacturing the same that are advantageous over this art.

### SUMMARY OF THE INVENTION

The disclosed invention is directed to a shaped collagen tissue implant and a method of manufacturing thereof. The properties of the shaped collagen tissue implant include improved self-adhesion, biocompatibility, flexibility, tissue integration, and compressibility over related products in the prior art. Following implantation of the shaped collagen tissue implant within a patient, the resultant tissue structure is not substantially changed over time because the implant serves as a biocompatible scaffold for the patient's own tissue regeneration.

The shaped collagen tissue implant of the invention is more biocompatible than currently produced implants due to the minimization, or exclusion, of chemical crosslinkers. Furthermore, the present method cuts and treats the collagen in a manner to provide a implant with an improved physical condition to permit host tissue integration, and eventual host tissue remodeling based on the void to fiber ratio, the drying/lyophilization conditions and biocompatibility. While not wanting to be bound by theory, it is anticipated that the host tissue remodeling of the implant occurs at a sufficiently slow rate, such that the tissue structure of the implant is preserved by the body. Alternatively, the remodeling of the implant can occur to such a complete extent that the implant structure is preserved in the body. Thus, the implant serves as a tissue expander and scaffold for the regrowth and remodeling, while maintaining the structure of the tissue.

An aspect of the invention is a shaped, collagen tissue implant. The implant includes a plurality of collagen pieces shaped to form the implant. The implant retains at least one dimension, a flexibility property, a cohesiveness property, or a compressibility property upon implantation into a patient.

An aspect of the invention is a method of forming a shaped, collagen-based implant. The method includes pretreating collagen to form treated collagen, processing the treated collagen to form collagen pieces, associating the collagen pieces in an aqueous solution to form a mixture, placing the mixture into a mold, and drying the mixture in the mold to form the implant.

### BRIEF DESCRIPTION OF THE FIGURES

FIG. 1 illustrates a perspective view of a shaped collagen tissue implant manufactured by the method of the invention; and

FIG. 2 illustrates the method of forming a shaped collagen tissue implant in accordance with the invention.

### DETAILED DESCRIPTION OF THE INVENTION

The invention relates to a shaped collagen tissue implant, and methods of making and using the same.

“Allogeneic” or “allograft”, as used herein, refers to tissue derived from a non-identical donor of the same species.

“Autogeneic” or “autograft”, as used herein, refers to tissue derived from and implanted into the same identical patient.

“Biocompatible”, as used herein, refers to the property of being biologically compatible with a living being by not causing harm.

“Biodegradable”, as used herein, refers to the property of being capable of being broken down by biological or environmental processes.

“Morselized”, as used herein, refers to the sectioning of a material into smaller pieces or fragments.

“Patient”, as used herein, refers to a living recipient of the biomaterial-based implants of the invention.

“Xenogeneic” or “xenograft”, as used herein, is defined as tissue derived from a non-identical donor of a different species.

An aspect of the invention is a shaped collagen tissue implant of specific dimensions. The implant includes a plurality of collagen pieces, where the average length of the collagen pieces is between about 1 and about 200 mm, and where the implant has between about 60% and about 100% of at least one post-manufacturing property. The post-manufacturing property can be at least one of a dimension, shape, re-hydrated flexibility, or re-hydrated compressibility.

The collagen of the tissue implant can be derived from tendons, ligaments, dermis, bone, articulating cartilage, connective tissue, dermal tissue, costal cartilage, fascia, dura matter, ligaments, tendons, pericardium, placental tissues, other collagens produced by plants or animals that are xenophobic in origin, or a combination of these tissue types. The collagen can be comprised solely of dermal tissue. The collagen of the tissue implant can be allogeneic, autogeneic, xenogeneic, or combinations thereof. The collagen pieces of the tissue implant can be a combination of collagen shapes or piece types, such as a mixture of collagen chunks and collagen fibers. In some embodiments, other materials can be entangled within or added to the collagen (e.g., antimicrobials, anti-inflammatories, bioactive minerals, biocompatible and biodegradable polymers, and stem cells). The amount of additional materials added to the collagen can range from about 1% to 75%, about 5% to 50%, or about 10% to 40%.

The shaped collagen tissue implant can be comprised of a single material or a mixture of materials, which can be used as scaffolding during tissue regrowth. In some embodiments, the collagen pieces can be combined with a second material. In some embodiments, the collagen pieces can be fully shaped and dehydrated and then coated with a second material. For example, the second material can be a mixture of a biodegradable polymer and an antimicrobial that is sprayed onto at least a portion of the surface of the shaped, collagen implant. In other embodiments, the collagen pieces can be combined with the second material in the course of the manufacturing process prior to the dehydration step. When the collagen pieces are combined with the second material during the manufacturing process, the materials can be more thoroughly entangled and homogenized prior to a final dehydration step. Examples of such second materials include, but are not limited to, another type of collagen, morselized cartilage injected into a demineralized bone matrix sponge, bone fibers encased in a biocompatible spinal implant, and morselized cartilage adhered to a bone plug surface to form an “osteochondral” implant. Suitable biocompatible spinal implants include, but are not limited to materials such as polyetheretherketone (PEEK), titanium, stainless steel, and combinations thereof. Suitable implants for collagen containment include, hollow cage implants, hollow screws, and other surgical implants with an internal void. Suitable implants for collagen encasement include the coverage of internal fixation pins, external fixation pins, wires, and so forth. Suitable demineralized bone matrix materials, including sponges, have been described in U.S. Pat. No. 8,574,825, which is incorporated in its entirety by reference. The amount of collagen-derived tissue in the shaped implant can range from about 25% to 99%, from about 50% to 95%, or from about 60% to 90%. When two sources of collagen are combined to form a composite implant, the ratio of the collagen sources can be about 10:1, about 5:1 or about 1:1.

Some embodiments can utilize tissue sources which match the desired tissue regeneration site. By including tissues structurally matched to the desired tissue regeneration site, the graft's ability to stimulate the specific tissue regrowth will be increased. For example, to repair a bone defect, an implant derived from bone collagen can be utilized. In the case of multiple tissue repair sites such as for osteochondral defects, osteochondral damage requires an implant that promotes repair of both the underlying subchondral bone and the chondral articulating surface. A composite, osteochondral allograft addresses the healing requirements of both cartilage and bone.

The collagen pieces for the invention can be generated by a variety of methods and techniques. For example, the collagen tissue can be shredded or cut into small pieces. In some embodiments, the collagen is shredded into fibers by cutting at an angle to the plane of the native collagen fibers, at an angle of about 30° to 90°, of about 45° to 90°, or of about 60° to 90° . Tools including graters, osteotomes, dermatomes, razers, slicers and scissors, can be used to prepare the collagen pieces.

Embodiments of the invention can include a collagen pretreatment step. The collagen can be decellularized. Methods to decellularize the collagen include, but are not limited to, demineralization, freeze-thaw cycling, cell lysis via hypotonic buffer, detergent soaks, nuclease soaks, and combinations of the preceding methods. In some embodiments, the native growth factors can be essentially completely or partially removed from the shaped collagen tissue implants. For example, the native growth factors within bone tissue can be depleted by soaking for a prolonged period of time in an acid solution (Pietrzak et al., BMP depletion occurs during prolonged acid demineralization of bone: characterization and implications for graft preparation, Cell Tissue Bank May;12(2):81-8 (2011)). For essentially complete removal of native growth factors, the quantity of growth factors can be below about one picogram per gram of the tissue. For partial removal of the native growth factors, the growth factor content can be about 5% to about 80%, about 10% to about 70%, about 15% to about 50% of the initial concentration as measured at the time of processing. In other embodiments, following essentially complete removal of the native growth factors, alternative growth factors can be added to the shaped collagen tissue implant prior to implantation (e.g., stem cells, fibroblast growth factors, vascular endothelial growth factors, and so forth). Suitable methods for adding the alternative growth factors include, but are not limited to soaking the implant in a solution containing the growth factors, spraying the implant with a solution containing the growth factors, injecting the implant with a solution containing the growth factors, and coating the implant with a paste of a mixture of growth factors in a liquid.

The presence of some growth factors within the shaped collagen tissue implant can promote a desirable level of implant integration within a patient. Desirable levels of implant integration can provide a projecting tissue body in a patient, which does not flatten or become misshapen over time. Additionally, following implantation of the shaped collagen tissue implant within a patient, the resultant tissue structure can retain its desired shape, flexibility, size, and texture.

The flexibility of the allograft can be further tailored to provide the ideal fit into a implantation site. For example, the allograft can conform to about 75% to about 100% of the void site. By fitting well into a defect, patient pain can be alleviated. Additionally, the ability of the implant to conform to the defect can provide for complete fill and the greatest opportunity for host tissue integration.

The collagen pieces of the invention can also be treated to provide optimal post-implantation physical characteristics. These optimal post-implantation physical characteristics included flexibility, texture, rigidity, size, shape, and compressibility. For flexibility, the hydrated implants can be bendable to about 90 degrees without loss of cohesion. For example, the texture of the implants can be smooth to permit smooth movement of an articulating joint. For a bone void, the texture of the implants can be irregular to promote osteoconductivity during the healing process. The rigidity and compressibility of the implants can be selected to provide the ideal pressure relief within a void site, such as realignment and decompression of spinal discs or complete fill of an osteochondral defect to alleviate pain. The size and shape of the implant will be sized to match the necessary implant site, such as a nipple reconstruction, a bone void, an osteochondral defect, or for a spinal fusion. The treatment can provide an implantation site that maintains the desired physical characteristics for a substantial duration of time following implantation into a patient. Methods to optimize post-implantation physical characteristics include: collagen crosslinking, protein denaturation, selective removal of growth factors, selective impregnation of growth factors, or introduction of additional implantation components. Collagen crosslinking can be achieved via chemical reactions, enzymatic treatment, or temperature exposure. For example collagen can be crosslinked by exposure to UV light, glycation, or heat. In some embodiments, the collagen can be cross-linked by soaking the collagen pieces in an aqueous solution at elevated temperature (e.g. soaked in water at about 40° C. to about 100 ° C. for about 2 hours). In some embodiments, the collagen can be cross-linked by subjected the collagen pieces to repeat freeze-thaw cycling. The implant can be a three-dimensional shape. The shape can be a cylinder, a cube, a block, a strip, a sphere, or any suitable shape for the desired application. The residual moisture content of the implant can be less than about 6%. The implant can be compressible to between about 40% and about 80% of its pre-compression size when between about 10 and 4000 grams-force/cm² is applied to the implant. In some embodiments, following compression the implant can return to its original shape of the pre-compression tissue base material. In some embodiments, the final shaped implant can remain greater than about 90% of the desired implant shape following manufacture. The implant can remain greater than 50% intact, in some embodiments greater than about 90% intact, after it is bent, compressed, twisted, squeezed or rolled. The implant can be bendable to about 90°. The implant can maintain at least one property, where the property can be shape, cohesiveness, pliability, or compressibility, for at least one year after manufacture.

Furthermore, the implant can maintain at least one dimension after implantation into a patient. In some embodiments, at least one dimension can be within about 80% to 100% of its pre-implantation dimension. The implant can also act as a natural host tissue after implantation into the patient.

The shaped collagen tissue implants of the invention have many advantages over the prior art. In some embodiments, the implant may not require rehydration to regain its pre-manufacturing flexibility, size, shape, and compressibility. In some embodiments, the rehydration in an aqueous fluid can aid the implant in regaining its pre-manufacturing flexibility, size, shape, and compressibility.

The implants of the invention can compress under a force of between about 10 grams-force/cm² and about 4000 grams-force/cm². The implants can be compressible to about 80% of their pre-compression size, to about 60% of their pre-compression size, to about 20% of their pre-compression size, to about 5% of their pre-compression size without loss of structural integrity or cohesion. Upon removal of an external compressing force, the implants can return to their pre-compression shape. In some instances, the shaped collagen tissue implants can also rehydrate rapidly within an aqueous fluid over a period of about 10 seconds to about 30 minutes, of about 1 minute to about 25 minutes, or of about 5 minutes to about 20 minutes. In some embodiments, the shaped collagen tissue implants can also have a high rehydration rate of between about 0.5 mL of fluid/g of implant/minute and about 10 mL of fluid/g of implant/minute. Suitable aqueous fluids include, but are not limited to, water, saline, buffer, balanced salt solution, blood, bone marrow aspirate, plasma and combinations thereof.

When the shaped, collagen-based implants of the invention are subjected to rehydration, the implants retain their shape, cohesiveness, pliability, and compressibility. The implants formed by the methods of the invention remain greater than about 50% intact in the dehydrated and rehydrated state, in some embodiments greater than 90% intact upon rehydration. The rehydrated implant can maintain a shape, cohesiveness, pliability, or compressibility for between about 1 hour and about 1 year after rehydration. The implants formed by the methods of the invention can be bendable to less than or equal to about 90°. The implants formed by the methods of the invention can maintain shape retention, cohesiveness, pliability, and compressibility post-manufacturing for time periods of about 1 year to about 5 years.

The implant can later be trimmed or otherwise adjusted to a specific shape, size, and/or appearance. The invention can also allow for the desired shape of the tissue implant to be manufactured without an additional post-forming step of trimming. The base material of the invention is collagen, which can be sized into fragments, chips, chunks, fibers, morsels, strips, particles, or combinations thereof. The void to collagen ratio of the implants can be determined and maintained because an external force may not be required during the formation of the implants of the invention.

In some embodiments, the shaped collagen tissue implant can contain voids. The void to collagen ratio can be controlled to between about 1:99 and about 1:1. In some embodiments, the void to collagen ratio can be between about 1:80 and about 1:1; about 1:70 and about 1:1; about 1:60 and about 1:1, or between about 1:30 and about 1:1. In some embodiments, the void to collagen ratio ranges from about 1: 19 and about 1:3.

An aspect of the invention is a method for forming a shaped collagen tissue implant of specific dimensions. The method includes pre-treating collagen to remove extraneous materials, shredding the collagen into pieces, entangling the collagen pieces in an aqueous solution, placing the entangled collagen pieces into a mold and drying the collagen in the mold. The resulting collagen tissue implant is three-dimensional of dimensions established by the use of a mold.

The method of manufacturing relies on judicious selection of collagen shapes and sizes, removal of extraneous material (e.g., fats), mixing with an aqueous liquid, molding, and drying of the shaped collagen tissue implant. Advantageously, the method is simple and inexpensive. In some embodiments, the shaped collagen tissue implant can be exposed to drying or lyophilization conditions. In some embodiments, collagen pieces can be shaped and sized to specific dimensions to enhance entanglement and subsequent final implant self-adhesion, flexibility, and compressibility. In some embodiments, the collagen pieces can be subjected to a tailored morselizing process in order to control the void to collagen ratio present in the manufactured shaped collagen tissue implant. The tailored morselizing process can consist of shaving pieces of the collagen source into sheets about 0.1 mm to 5 mm thick, 0.1 mm to 10 mm wide, and about 0.1 mm to 10 mm long. In other embodiments, the tailored morselizing process can consist of longer strips of the collagen source into pieces of about 0.1 mm to 5 mm thick, 0.1 mm to 5 mm wide, and about 1 mm to 200 mm long. By controlling the void to collagen ratio of the implant, the post-implantation tissue structure can retain its desired shape, flexibility, appearance, and texture.

The drying/lyophilization conditions allow for an enhanced implant, which does not require chemical crosslinking agents. Following forming of the collagen pieces into the mold, the filled mold can be frozen at a temperature of about −100° C. to about 0° C., of about −90° C. to about −10° C. to about −80° C. to about −20° C. The apparatus can be placed into a drying chamber in a frozen or thawed state. The drying step can include subjecting the apparatus to reduced pressure, heating, lyophilization (under reduced pressure), or a combination of dehydration and lyophilization. In preferred embodiments, the drying/lyophilization step can be performed under reduced pressures of about 100 Torr to about 600 Torr, about 200 Torr to about 400 Torr, about 300 Torr. Drying can include heating the material to a temperature between about 30° C. and about 80° C., in some embodiments about 45° C. The temperature of the drying step can change over the time period of the drying from about −80° C. to 80° C., from about −70° C. to 60° C., from about 0° C. to 50° C., from about 25° C. to 45° C. Drying can take place over the range of about 1 hour to about 48 hours, of about 3 hours to about 24 hours, of about 4 hours to about 20 hours.

During the drying step, the vacuum can be increased from an initial pressure of about 100 Torr to about 600 Torr, of about 200 Torr to about 400 Torr, of about 300 Torr to about 2500 mTorr to about 200 mTorr, about 2000 mTorr to about 300 mTorr, or about 1000 mTorr.

Utilizing the drying/lyophilization conditions of the invention, the final shape of the implant is within about 10% of at least one dimension of its projected size based on the mold, and dimensions of the mold. The shaped collagen tissue implants can be shaped in the form of a cylinder, cube, strip, sphere, or other three-dimensional shape as desired. The shape of the implants can be uniform or irregular as desired by the end-user of the article. In some embodiments, the size of the shaped collagen tissue implant can be larger than the final desired implant and cut to a desired dimension.

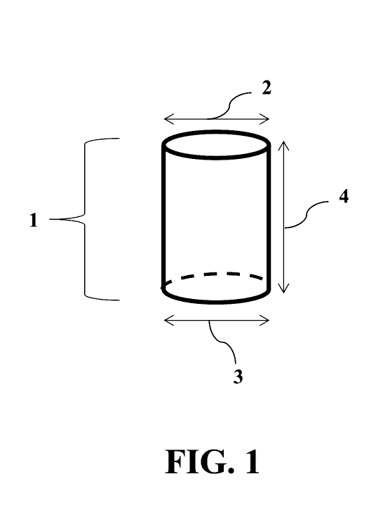

As illustrated in FIG. 1, in some embodiments the shaped collagen tissue implant can be in the form of a cylinder 1. The diameter 2 of the shaped collagen tissue implant can range from about 1 and about 200 mm, about 2 and about 100 mm, or about 5 and about 50 mm. The diameter 3 of the shaped collagen tissue implant can range from about 1 and about 200 mm, about 2 and about 100 mm, or about 5 and about 50 mm. The diameters 2 and 3 can be the same or different. One skilled in the art would understand that if the implant was a block, strip or cube, the width can be the same dimensions as the diameter 2, the length can be the same dimensions as the diameter 3 without deviating from the invention. The height 4 of the tissue implant can range from about 1 and about 200 mm, about 2 and about 100 mm, or about 5 and about 50 mm.

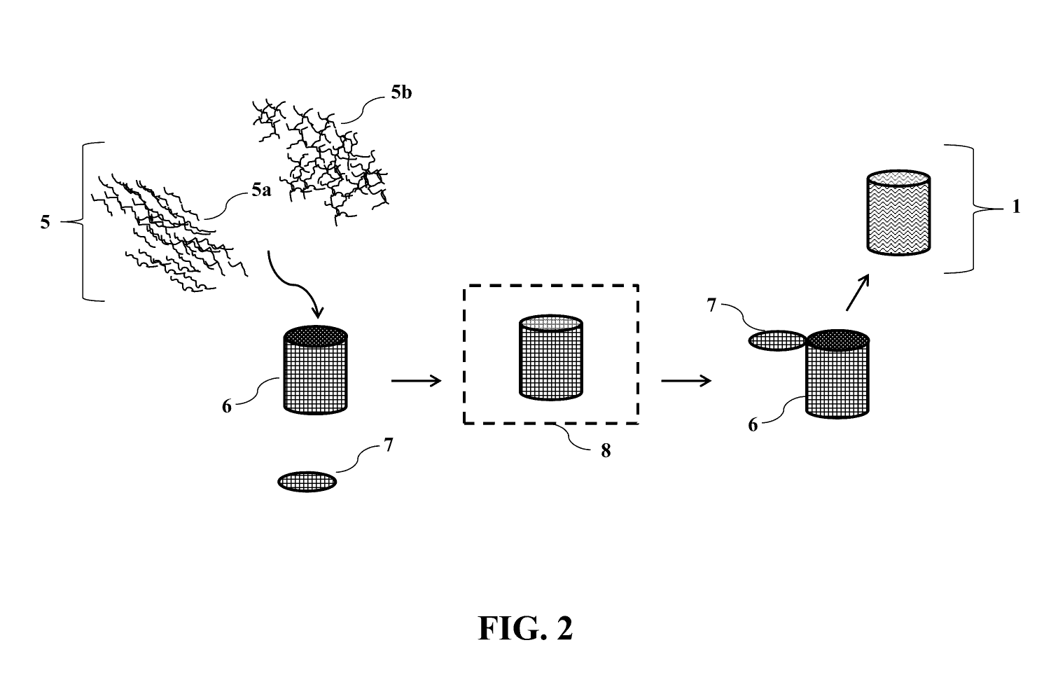

As illustrated in FIG. 2, the collagen pieces 5 of the shaped collagen tissue implant are formed from collagen pieces in a regular 5a or irregular arrangement 5b. In some embodiments, the collagen pieces can be placed in a consistent, parallel orientation 5a. In some embodiments, the collagen pieces of the shaped collagen tissue implant can be entangled and interlaced in multiple orientations 5b to provide strength and adhesion of the collagen pieces to one another within the resultant implant. The collagen pieces within the tissue implant 1 can be of lengths of about 1 mm to about 200 mm, of about 2 mm to about 150 mm, of about 5 mm to about 70 mm, to about 10 mm to about 60 mm. The collagen pieces can have diameters of about 0.1 mm to about 30 mm, of about 0.2 mm to about 15 mm, of about 0.5 mm to about 10 mm, to about 1 mm to about 8 mm. It is known in the art that implant porosity affects host integration post-implantation. Accordingly, judicious selection of the size and shape of the collagen pieces during manufacture can allow for control of the implant's collagen surface area and porosity for improved integration with host tissue.

Following any necessary pre-treatment (e.g., defatting, decellularizing, and demineralizing), the collagen pieces 5 can be placed within a mold 6 as illustrated in FIG. 2. Following forming of the collagen pieces into the mold 6, the filled mold can be frozen at a temperature of about −100° C. to about 0° C., of about −90° C. to about −10° C. to about −80° C. to about −20° C. The mold 6 can be placed into a drying chamber 8 in a frozen or thawed state.

The mold 6 illustrated in FIG. 2 is capable of forming a three-dimensional shape. At least one dimension of the mold can be selected to result in a shape to manufacture an implant to correspond with a particular void. In some embodiments, the mold can correspond to a damaged structure. The mold can be manufactured from a three-dimensional printer. In some embodiments, the mold can fully enclose the collagen pieces, or can have a lid 7 if desired. The lid 7 can be attached to the mold, detachable, or separate from the mold. The mold 6 and lid 7 can be perforated to fully or partially to allow for the removal of moisture from the bone pieces 5 during shaping. In other embodiments, the mold can be used to form a three-dimensional shape, for example, a tube of material, which can be further shaped. The mold 6 can be composed of various heat resistant materials such as, but not limited to, ceramics, elastomers, aluminum, stainless steel, thermoplastics, any other metals, or combinations thereof. The mold 6 can be amenable to steam sterilization and elevated temperatures, for example between about 30° C. and 80° C. The mold 6 dimensions can be pre-set or adjustable to the desired final implant dimensions. The mold 6 can be constructed of a screen-like material. The mold 6 can have a non-stick coating, such as Teflon. The mold 6 or mold lid 7 can apply adjustable inward pressure.

In some embodiments, the mold 6 can have drainage holes or openings to allow moisture to enter and exit the tissue during use of the mold 6. In some embodiments, the mold 6 can have openings or drainage holes at least on one side. In another embodiment, the mold can be comprised of only three sides so that moisture can exit from open sides of the mold 6. In another embodiment, the mold 6 can be comprised of a screen with numerous openings to allow moisture entry or exit during use. In other embodiments, the mold 6 can be a sieve or strainer. The shaped collagen tissue implant can be used to produce a shaped collagen tissue body on a patient. The size and shape of the shaped collagen tissue body can be selected by selection of the shaped collagen tissue implant of the required dimensions. In some embodiments, the shape can mimic a naturally occurring shape, such as a body part. By way of example only, in some embodiments, the final use of the shaped collagen tissue implant can be use for nipple reconstruction.

In some embodiments, the size and shape of the collagen tissue implant can be customized for alternative locations of implantation and corresponding tissue structures of a patient. Examples include, but are not limited to, a shaped implant corresponding to a human ear, a shaped implant corresponding to a human nose, a shaped implant corresponding to a soft tissue defect, a shaped implant customized for used in breast augmentation surgery, or a shaped implant customized for use in for a phalloplasty surgery. In these examples, the shaped collagen tissue implant could be used for customized reconstruction or plastic surgeries yielding improved aesthetic appearance compared to traditional plastic and reconstructive surgical techniques.

During the forming of the shaped collagen tissue implant 1, the collagen pieces can be laid in a regular pattern 5a within the mold 6, or entangled as a mesh, braid, or interwoven in some manner 5b prior to placement within the mold 6. In some embodiments, the collagen pieces can be laid or entangled around another article. If the collagen pieces are placed around another article, the contained article can be comprised of the same material or of materials of a different composition than the surrounding collagen pieces (e.g., a shaped bone fiber-based article composed of demineralized cancellous bone fibers can be placed inside bone particles composed of demineralized cortical bone). The contained article can be composed of elastomers, ceramics, metals, metal alloys, or plastics. In some embodiments, the surrounding collagen pieces can be impregnated with an alternative collagen source, such as morselized cartilage.

The mold with the material can be dried in a drying chamber 8. Following drying, the mold can be removed from the drying chamber 8, and the shaped collagen tissue implant 1 can be removed from the mold 6. In some embodiments, the drying of collagen pieces 5 within the mold 6 under the conditions described results in an article with improved shape retention and enhanced cohesiveness and flexibility upon rehydration. In the preferred embodiments, the drying of the collagen pieces 5 within the mold 6 under the conditions described results in an article with desired level of growth factors and a residual moisture content of less than about 6%, less than about 4%, or less than about 2%.

### EXAMPLE

### Method of Preparing a Composite, Shaped Collagen Implant

A section of human cancellous bone was demineralized with 0.6 N HCl, using the demineralization method disclosed in U.S. Pat. No. 9,114,191 Process for Demineralization of Bone Matrix with Preservation of Natural Growth Factors, which is incorporated herein by reference in its entirety. Concurrent to the bone demineralization, sections of articulating cartilage were morselized into pieces sized between about 1 mm and about 4 mm. The morselized cartilage was manually injected into the porous voids within the demineralized cancellous bone. After combination of the two collagen types in a 10:1 ratio of bone to cartilage, the composite material was then dried to provide enhanced adherence of the cartilage to the bone matrix. The implant was dried first at 45° C. at 760 torr for 60 minutes, followed by 35° C. at 2450 mtorr for 720 minutes. The resultant graft demonstrated compressibility within about 90% of its initial compressibility using between about 10 and 1000 grams-force/cm². Furthermore, within about 90% of the tissue components remained intact after rehydration in a saline solution.

Ranges have been discussed and used within the forgoing description. One skilled in the art would understand that any sub-range within the stated range would be suitable, as would any number within the broad range, without deviating from the invention.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A shaped, collagen tissue implant, comprising:
a plurality of collagen pieces, wherein the plurality of collagen pieces are shaped to form the implant, wherein the implant retains at least one of at least one dimension, a flexibility property, a cohesiveness property, or a compressibility property upon implantation into a patient.

2. The shaped, collagen tissue implant of claim 1, wherein an average length of the pieces is between about 1 mm to about 200 mm.

3. The shaped, collagen tissue implant of claim 1, wherein an average diameter of the collagen pieces is between about 0.1 mm to about 30 mm.

4. The shaped, collagen tissue implant of claim 1, wherein a shape of the implant is selected from the group consisting of a cube, a cylinder, a block, a strip, and a sphere.

5. The shaped, collagen tissue implant of claim 1, wherein the at least one specific dimension of the implant correspond to a naturally occurring tissue structure.

6. The shaped, collagen tissue implant of claim 1, wherein a source of the collagen is selected from the group consisting of an articulating cartilage, a connective tissue, a dermis, a costal cartilage, a fascia, a dura matter, a ligament, a tendon, a pericardium, a placental tissue, a demineralized bone matrix and combinations thereof.

7. The shaped, collagen tissue implant of claim 1, wherein the implant is composed of at least two collagen sources.

8. The shaped, collagen tissue implant of claim 7, wherein the at least two collagen sources is a bone and a cartilage.

9. The shaped, collagen tissue implant of claim 1, wherein the plurality of collagen pieces are decellularized.

10. The shaped, collagen tissue implant of claim 1, wherein a material of the plurality of collagen pieces is selected from the group comprising an allogeneic tissue, an autogeneic tissue, a xenogeneic tissue, and combinations thereof.

11. The shaped, collagen tissue implant of claim 1, wherein the implant is dehydrated to form a dehydrated implant.

12. The shaped, collagen tissue implant of claim 11, wherein the dehydrated implant rehydrates in at least one aqueous liquid in between about 10 seconds and about 30 minutes.

13. The shaped, collagen tissue implant of claim 11, wherein the dehydrated implant remains greater than about 50% intact upon rehydration to form a rehydrated implant.

14. The shaped, collagen tissue implant of claim 13, wherein the rehydrated implant maintains at least one property of at least one dimension, a cohesive property, a flexibility property, and a compressibility property for between about 1 hour to about 1 year after rehydration.

15. The shaped, collagen tissue implant of claim 1, wherein the implant is bendable to less than or equal to about 90°.

16. The shaped, collagen tissue implant of claim 1, wherein the implant is compressible to about 40% to about 80% of a size after manufacturing.

17. The shaped, collagen tissue implant of claim 1, wherein the implant returns to a pre-compression shape following a compression of the implant.

18. The shaped, collagen tissue implant of claim 1, wherein the implant remains greater than about 50% intact after at least one procedure selected from the group consisting of bending, compressing, twisting, squeezing, and rolling.

19. A method of forming a shaped, collagen-based implant, comprising:
pretreating collagen to form treated collagen;
processing the treated collagen to form collagen pieces;
associating the collagen pieces in an aqueous solution to form a mixture;
placing the mixture into a mold; and
drying the mixture in the mold to form the implant.

20. The method of claim 19, wherein the mold forms the implant into a shape selected from the group consisting of a cube, a cylinder, a block, a strip, and a sphere.

21. The method of claim 19, wherein the collagen is selected from the group consisting of an articulating cartilage, a connective tissue, a dermis, a costal cartilage, a fascia, a dura matter, a ligament, a tendon, a pericardium, a placental tissue, a demineralized bone matrix and combinations thereof.

22. The method of claim 19, wherein the collagen pieces are substantially decellularized.

23. The method of claim 19, wherein the mold is manufactured by three-dimensional printing.

24. The method of claim 19, wherein at least one dimension of the mold is selected to fill a void.

25. The method of claim 19, wherein at least one dimension of the mold is selected replace a damaged tissue structure.

26. The method of claim 19, wherein the drying step heats the mold to a temperature between about 30° C. and about 80° C.

27. The method of claim 19, wherein the drying step is lyophilization.
